(19) United States
(12) Patent Application Publication
(10) Pub. No.: US 2025/0086350 A1
(43) Pub. Date: Mar. 13, 2025

(54) METHOD FOR COMPREHENSIVELY APPORTIONING THE SOURCE CONTRIBUTION TO PM2.5 BASED ON THE RECEPTOR MODEL AND THE CHEMICAL TRANSPORT MODEL

(71) Applicant: Chongqing Research Academy of Eco-Environmental Sciences, Chongqing (CN)

(72) Inventors: Zhenliang LI, Chongqing (CN); Yunqing CAO, Chongqing (CN); Yuhong QIAO, Chongqing (CN); Chao PENG, Chongqing (CN); Weikai FANG, Chongqing (CN); Xiaochen WANG, Chongqing (CN); Linfeng DUAN, Chongqing (CN); Mulan CHEN, Chongqing (CN); Min DU, Chongqing (CN); Sheng ZHANG, Chongqing (CN)

(21) Appl. No.: 18/755,377

(22) Filed: Jun. 26, 2024

(30) Foreign Application Priority Data
Sep. 8, 2023 (CN) 202311157836.8

(51) Int. Cl. G06F 30/20; G01N 33/00

(52) U.S. Cl. CPC G06F 30/20; G01N 33/0037; G01N 33/0042; G01N 33/0047

(57) ABSTRACT

This invention discloses a method for comprehensively apportioning the source contribution to fine particular matter (PM_{2.5}) based on the receptor model and the chemical transport model, which comprises the receptor model calculation steps, chemical transport model calculation steps and comprehensive source apportionment steps; the said comprehensive source apportionment step comprises the following sub-steps: according to the principle of inverse proportionality between uncertainty and weight coefficient, the first (receptor model) uncertainty and the second (chemical transport model) uncertainty are normalized to obtain their respective weight coefficients; the comprehensive source apportionment results are calculated based on the apportionment results of the receptor model and the chemical transport model, and their respective weight coefficient. The invention integrates the respective advantages of the receptor model and chemical transport model by using weight coefficients obtained through the normalization of uncertainties, thus improving the accuracy and reliability of the source apportionment result of PM_{2.5}.

### TECHNICAL FIELD

This invention relates to the technical field of air quality value simulation, in particular to a method for comprehensively apportioning the source contribution to The fine particulate matter (PM_{2.5}) based on the receptor model and the chemical transport model.

### BACKGROUND TECHNOLOGY

The fine particulate matter (PM_{2.5}) is an important evaluation index of air quality in China. Excessive PM_{2.5} concentration will be very harmful to the atmospheric environment and human health and is the main problem in preventing and controlling air pollution in China. The technology of qualitative or quantitative research on the sources of atmospheric particulate matter is called source apportionment technology. The source apportionment results are the basis for formulating air pollution prevention and control plans and are of great guiding significance for determining the key points of pollution control. The source apportionment method of urban particulate matter is generally divided into two categories: the receptor model method and the air quality model method.

The receptor model aims to study the contribution of the emission sources to receptors. The so-called receptor refers to a local atmospheric environment studied relative to the emission source. The receptor model is used to qualitatively identify the pollution sources that contribute to the receptors and quantitatively calculate each pollution source's sharing rate by measuring the sources' physical and chemical properties and atmospheric environment (receptor) samples. The receptor model has diversified types, mainly including chemical mass balance (CMB), principal factor analysis (PFA), multiple linear regression analysis (MLR) and target transformation factor analysis (TTFA). Among them, the CMB model provides a clear physical significance, and has become the most important and practical receptor model with the increasingly mature algorithm. CMB is composed of a set of linear equations, and refers to the linear sum of the products of the content value of each chemical component in the composition spectrum of each emission source that corresponds to the receptor concentration of the chemical component and the contribution concentration value of the receptor. However, CMB can only be used to consider the primary pollution contribution but cannot simulate the secondary pollution contribution of the particulate matter.

The chemical transport model is employed to simulate the processes of particulate matter transport, reaction, and removal in the atmosphere. This is accomplished by leveraging the scientific understanding of physical and chemical processes in the atmosphere, coupled with the application of meteorological principles and mathematical methods. Through investigation, the source intensity distribution of the anthropogenic emission source is obtained, and the concentration contribution of each source to any control point at the control area can be estimated by inputting it into the chemical transport model. CAMx model (chemical transport model) is one of the mainstream models used in air quality simulation, which synthesizes all the technical features required by the “Scientific” air quality model into a single system and can be used to comprehensively evaluate gaseous and particulate air pollutants at various urban and regional scales. Its particulate matter source apportionment technology (PSAT) could track the contribution of the source to the simulated particle matter concentrations through the source area and/or category. For the CAMx/PSAT model, the influence of secondary reaction is fully considered, but its traceability results may have a large error due to the lag and uncertainty of emission inventory.

In conclusion, the receptor model and the chemical transport model method have their own advantages and limitations in the process of atmospheric source apportionment, and both of them can improve the accuracy and reliability of the apportionment result because they can be applied to comprehensively apportion the sources of PM2.5.

### Contents of the Invention

The purpose of this invention is to overcome the shortcomings of the prior art and provide a method for comprehensively apportioning the source contribution to PM_{2.5} based on the receptor model and the chemical transport model.

The purpose of this invention can be achieved through the following technical schemes:

In the first aspect, this invention provides a method for comprehensively apportioning the source contribution to PM2.5 based on the receptor model and the chemical transport model, including a receptor model calculation step, a chemical transport model calculation step and a comprehensive source apportionment step;

The said receptor model calculation step comprises the following sub-steps:

According to the receptor points, the process involves setting up a simulation grid, compiling a grid-based emission inventory, and simulating the meteorological field using the mesoscale numerical weather forecast model WRF;

Implementing receptor sampling analysis to obtain the component concentration of PM_{2.5} at the receptor points, and inputting the component concentration into the receptor model CMB to obtain the primary pollution contribution of different sources to PM_{2.5};

Based on the said meteorological field, using the potential source contribution calculation method PSCF in the backward trajectory model to obtain the spatial range having potential source influence on the concentration of PM_{2.5}; calculating the emission proportions of sulfur dioxide, nitrogen oxides and volatile organic compounds from different sources in the spatial range;

Distributing the pollution source contribution to the secondary component concentration of PM_{2.5} according to the said emission proportion to obtain the secondary pollution contributions of different sources to PM_{2.5}; Adding the primary pollution contribution of different sources to PM_{2.5} and the secondary pollution contribution of different sources to PM_{2.5}, so as to obtain the overall pollution contribution of different sources to PM_{2.5}, i.e., the source apportionment result SRⱼ of the receptor model;

Calculating the first uncertainty σⱼ^{SR} of the source apportionment result SRⱼ of the receptor model;

The said chemical transport model calculation step comprises the following sub-steps:

According to the receptor points, setting up a simulation grid, compiling a grid-based emission inventory, and simulating the meteorological field using the mesoscale numerical weather forecast model WRF;

Inputting the said meteorological field and the grid-based emission inventory into the chemical transport model CAMx/PSAT to obtain the initial simulated concentration of the PM_{2.5} components and the pollution source contribution value of PM_{2.5};

The optimization solution is performed based on the least-squares error between the simulated result and the observed result of the chemical transport model of the PM_{2.5} component concentration at the receptor point, so as to obtain the correction factor for the simulated result of the chemical transport model;

Using the said correction factor to correct the value of the pollution source contribution to PM_{2.5} simulated by the chemical transport model, so as to obtain the corrected value of the pollution source contribution to PM_{2.5}, i.e., the apportionment result of the chemical transport model SAⱼ;

Calculating the second uncertainty σⱼ^{SA} of the apportionment result SAⱼ of the chemical transport model;

The said comprehensive source apportionment step comprises the following sub-steps: The uncertainty range of the model apportionment result represents the error range of the result, so the range of the first uncertainty σⱼ^{SR} and the range of the second uncertainty σₕ^{SA} are normalized according to the principle that the uncertainty range is inversely proportional to the weight coefficients, so as to obtain their respective weight coefficient. The calculation formula is as follows:

$$\{ \begin{matrix}{w}_{j}^{SR} = \frac{span ( {σ}_{j}^{SA} )}{span ( {σ}_{j}^{SR} ) + span ( {σ}_{j}^{SA} )} \\ {w}_{j}^{SA} = \frac{span ( {σ}_{j}^{SR} )}{span ( {σ}_{j}^{SR} ) + span ( {σ}_{j}^{SA} )}\end{matrix}$$

Wherein, wⱼ^{SR} and wⱼ^{SA} respectively represent the weight coefficient of the source apportionment result of the receptor model and the weight coefficient of the source apportionment result of the chemical transport model, and “span” represents the uncertainty range calculated;

Calculating the comprehensive source apportionment result Sⱼ based on the source apportionment result SRⱼ of the receptor model, weight coefficient wⱼ^{SR} of the source apportionment result of the receptor model, apportionment result SAⱼ of the chemical transport model and weight coefficient wⱼ^{SA} of the source apportionment result of the chemical transport model:

$${S}_{j} = {w}_{j}^{SR} \times {SR}_{j} + {w}_{j}^{SA} \times {SA}_{j} .$$

Further, setting up a simulation grid according to the receptor points as said above, comprising:

The receptor points are the points to be traced for sources, the simulation grid is a WRF simulation grid, and the simulation grid of the study area should cover the receptor points;

Compiling a grid-based emission inventory as said above, comprising:

Inputting the products listed on the local anthropogenic emissions or other public emission inventory calculated by using the factor accounting method based on the data, including environmental statistics, pollutant discharge permit and enterprise research into the emission inventory processing model SMOKE, so as to obtain the grid-based pollutant discharge inventory suitable for the chemical transport model CAMx/PSAT.

Further, simulating the meteorological field using the mesoscale numerical weather forecast model WRF as said above, comprising:

Inputting the re-analysis meteorological data, local terrain elevation and data of underlying surface covered by land into WRF, simulating the meteorological field for a period of time, and verifying the simulated result and optimizing its parameters based on the observed data of the meteorological station.

Further, implementing receptor sampling analysis to obtain the component concentration of PM_{2.5} at the receptor points mentioned above, comprising:

Collecting the particulate filter membrane using an atmospheric sampler, the chemical element analysis, carbon analysis and ion analysis of the samples are completed using the inductively coupled plasma mass spectrometer (ICP-MS), inductively coupled plasma spectrometry (ICP-OES), ion chromatography, and thermal/optical carbon analyzer; The analyzed components comprise one or more of the chemical elements below: Li, Be, Na, P, K, Sc, As, Rb, Y, Mo, Cd, Sn, Sb, Cs, La, V, Cr, Mn, Co, Ni, Cu, Zn, Ce, Sm, W, TI, Pb, Bi, Th, U, Zr, Al, Sr, Mg, Ti, Ca, Fe, Ba, Si; one or more of the carbon components below: TC, OC and EC, and one or more of the ion components below: Na⁺, Mg²⁺, Ca²⁺, K⁺, NH₄⁺, SO₄²⁻, Cl⁻ and NO₃⁻;

The said different sources comprise power sources, industrial sources, traffic sources, domestic sources, agricultural sources and other sources.

Further, based on the said meteorological field, using the potential source contribution calculation method PSCF in the backward trajectory model to obtain the spatial range having potential source influence on the concentration of PM_{2.5}; calculating the emission proportions of sulfur dioxide, nitrogen oxides and volatile organic compounds from different sources in the spatial range, as said above, comprising:

Converting the simulated result WRFOUT file of WRF into a format that can be recognized by HYSPLIT model through HYSPLIT model pre-processing tool, then inputting the converted meteorological data into HYSPLIT model for simulation to obtain a backward trajectory for a period of time, and finally obtaining the spatial range having potential source influence and the PSCF value of each grid through PSCF method based on the backward trajectory;

PSCF is a method to characterize the pollution contribution of each grid to the receptor points by using the probability of airflow traceability. This method can divide the study area into several small horizontal grids based on longitudes and latitudes and set the concentration threshold of the pollutants to compare whether the pollutant concentrations at the traceability points in the grids are higher than the threshold (for example, the concentration of PM_{2.5} is higher than 35 μg/m³) and thus determine the number of pollution trajectory points in the grids. The PSCF value is the ratio of the number (mᵢⱼ) of pollution trajectory points passing through the grid (i, j) in the study area to the number (nᵢⱼ) of all trajectory points passing through the grid, namely PSCF=mᵢⱼ/nᵢⱼ.

The total amount of emission of a certain type of pollutant from a certain source is calculated by

$$E = \frac{\sum ( {PSCF}_{ij} \times {E}_{ij} )}{\sum {PSCF}_{ij}}$$

Wherein, Eᵢⱼ represents the emission of the pollutants from the source in (i, j) grid. Accordingly, the emissions of sulfur dioxide, nitrogen oxides and volatile organic compounds from different sources that affect the receptor points and the proportion of the total emissions of such pollutants from all sources in the spatial range can be calculated.

Further, calculating the first uncertainty σⱼ^{SR} of the source apportionment result SRⱼ of the receptor mode, as said above, comprising:

The first uncertainty σⱼ^{SR} is calculated based on the observed error and the emission inventory error of the PM_{2.5} components:

$${σ}_{j}^{SR} = {\sum }_{i = 1}^{N} ( {p}_{i} {σ}_{i , j}^{obs} + {p}_{i}^{′} {σ}_{i , j}^{emi} )$$

Wherein, pᵢ is the concentration proportion of the primary component i of PM_{2.5}; σ_{i,j}^{obs} is the observed error of the PM_{2.5} component, which is specifically reflected on the observed uncertainty of the PM_{2.5} component i and source j; p′ᵢ is the concentration proportion of the secondary component i of PM_{2.5}; σ_{i,j}^{emi} is the emission inventory error, which is specifically reflected on the uncertainty of the precursor emission inventory of the secondary component i and source j of PM_{2.5};

Among them, the observed error σ_{i,j}^{obs} of the PM_{2.5} component is calculated based on the ratio of the maximum permissible error to the typical observed concentration of the component observation instrument; the emission inventory error σ_{i,j}^{emi} is calculated based on the uncertainty of the multiscale air pollution emission inventory.

Further, by inputting the said meteorological field and the grid-based emission inventory into the chemical transport model CAMx/PSAT, the main parameterization schemes are specifically as follows: the initial field and boundary conditions are ICBCPREP, the meteorological chemical mechanism is CB05, the aqueous chemical mechanism is RADM, the aerosol scheme is the CF scheme, the secondary organic chemical scheme is SOAP, the aerosol thermodynamic equilibrium model is ISORROPIA, the dry deposition parameterization scheme is ZHANG03, the horizontal advection scheme is the PPM scheme, and the vertical diffusion scheme is the standard K theory. Further, performing optimization solution based on the least-squares error between the simulated result and the observed result of the chemical transport model of the PM_{2.5} component concentration at the receptor point, so as to obtain the correction factor of the simulated result of the chemical transport model, as said above, comprising:

Taking the least-squares error between the CAMx simulated concentration and observed concentration of the PM_{2.5} component at the receptor point as the target function to obtain the correction factor corresponding to the minimum error, with the target function below:

$$min {\sum }_{i = 1}^{N} [ \frac{{( {C}_{i}^{obs} - {C}_{i}^{sim} - {\sum }_{j = 1}^{M} {SA}_{i , j}^{base} ( {R}_{j} - 1 ) )}^{2}}{{σ}_{i , obs}^{2} + {σ}_{i , sim}^{2}} ]$$

$$s . t . 0.1 \leq {R}_{j} \leq 20$$

Wherein, Rⱼ is the correction factor of the model apportionment result of the source j; Cᵢ^{obs}, Cᵢ^{sim} are respectively the observed concentration value and the simulated concentration value of the component i; SA_{i,j}^{base} is the simulated contribution concentration of the initial model of the component i and the source j; σ_{i,obs}, σ_{i,sim} are respectively the uncertainties of the observed concentration and the simulated concentration of the component i;

Inputting the observed result of the PM_{2.5} component and the initial source contribution of PSAT, etc., into the target function, and performing a non-linear optimization solution on the target function to obtain the correction factor R of the model apportionment result; using the step-by-step iterative optimization calculation method in the solution procedure, that is, the initial iterative optimization step size is 0.5, and the optimization step size of 0.01 is adopted after the preliminary determination of range.

Further, as said above, using the said correction factor to correct the value of the pollution source contribution to PM_{2.5} simulated by the chemical transport model, so as to obtain the corrected value of the pollution source contribution to PM_{2.5}, i.e., the apportionment result of the chemical transport model SAⱼ, with the calculation formula comprising:

$${SA}_{i , j}^{adj} = {R}_{j} \times {SA}_{i , j}^{base}$$

$${SA}_{j} = {\sum }_{i = 1}^{N} {SA}_{i , j}^{adj}$$

Wherein, SA_{i,j}^{adj} is the simulated contribution concentration of the correction model of the PM_{2.5} component i and source j; SAⱼ is the simulated source apportionment result of the PM_{2.5} model of the source j.

Further, calculating the second uncertainty σⱼ^{SA} of the apportionment result SAⱼ of the chemical transport model, as said above, with the calculation formula comprising:

$${σ}_{j}^{SA} = {\sum }_{i = 1}^{N} {p}_{j} f ( {σ}_{i , j}^{emi} )$$

Wherein, pᵢ is the concentration proportion of the PM_{2.5} component i; σ_{i,j}^{emi} is the uncertainty of the emission inventory of the PM_{2.5} component i and source j, and f (σ_{i,j}^{emi}) is the uncertainty of the simulated result derived from that of the emission inventory.

This invention has brought the beneficial effects below: in an exemplary embodiment of this invention:
(1) Calculating the weight coefficients of the two source apportionment results based on the normalization of the uncertainties of the receptor model and the chemical transport model, so as to obtain the comprehensive source apportionment result; the effects are as follows:
(1-1) Compared with the prior art, which is very complicated to calculate the multi-model data similarity to obtain the model weight coefficient. In this exemplary embodiment, the model weight coefficient is obtained through the uncertainty, in which the uncertainty is used to evaluate the error of the source apportionment result of the model (which can be directly extracted from the database for use in some cases) as a parameter that can be easily calculated after the source apportionment result of each model is calculated, so it is unnecessary to calculate additional parameters in the common process (additional parameters are required to be calculated in calculating the data similarity in the prior art) to reduce the amount of data calculated;
(1-2) At the same time, the uncertainty of the model apportionment result represents the resulting error, and the uncertainty can be a positive deviation or negative deviation in some cases, so the uncertainty range (i.e., the difference between the positive deviation and the negative deviation of the uncertainty) can characterize the possible deviation range of the results. After the first uncertainty range and the second uncertainty range are normalized based on the principle that the uncertainty range is inversely proportional to the weight coefficient (a large range corresponds to a small weight, while a small range corresponds to a large weight), so as to obtain their respective weight coefficient, thus improving the accuracy and reliability of the source apportionment result of PM_{2.5};
(1-3) In addition, the weight coefficients of all sources (industrial sources, traffic sources, domestic sources, etc.) are determined based on their respective uncertainty result to provide higher purposefulness and refinement (because different sources have different pollutant components and characteristics, the receptor model and the chemical transport model are different in terms of the applicability), and more refined traceability results can be obtained through the differentiated source contribution weight coefficients.
(2) For the receptor model, the potential source contribution analysis method is used to specify the influence range of the pollution transport of PM_{2.5}, and the source contribution to the secondary component of PM_{2.5} is distributed and calculated based on the precursor emissions proportions of all resources based on the range above, thus optimizing the source apportionment result of the secondary component of the receptor model; while for the chemical transport model, the least-squares error between the simulated result and the monitoring data of the PM_{2.5} components is used to optimize the model and thus correct the source apportionment result of the chemical transport model.

### DESCRIPTION OF ATTACHED FIGURES

FIG. 1 is the flow chart of a method for comprehensively apportioning the source contribution to PM_{2.5} based on the receptor model and the chemical transport model provided in an exemplary embodiment of this invention;

FIG. 2 is the schematic diagram of a simulated grid provided in an exemplary embodiment of this invention;

FIG. 3 is the schematic diagram of the potential source influence of a receptor point provided in an exemplary embodiment of this invention;

FIG. 4 is the schematic diagram of the optimization simulation error (a) and the distribution range of the pollution source contribution correction factor (b) of the chemical transport model CAMx/PSAT provided in an exemplary embodiment of this invention.

### EMBODIMENTS OF THIS INVENTION

The technical proposals of this invention are clearly and completely described below in combination with the attached figures. Obviously, only a part not all of the embodiments of this invention are given here. Based on the embodiments of this invention, all other embodiments obtained by those who are ordinarily skilled in the art without creative works are within the scope of protection of this invention.

In the description of this invention, it should be noted that terms indicating direction or positional relations such as “center”, “upper”, “lower”, “left”, “right”, “vertical”, “horizontal”, “inner” and “outer” are given based on the attached figures to facilitate and simplify the description of this invention. These terms are not meant to indicate or imply that the devices or elements mentioned must have specific orientations and be constructed and operated based on specific orientations, so they should not be constructed as limitations of this invention. In addition, the terms “first” and “second” are only used for descriptive purposes, but should not be constructed to indicate or imply the relative importance.

In the description of this invention, it should be noted that unless otherwise expressly stipulated and limited, the terms “Installation”, “Connected” and “Connection” should be broadly understood. For example, they may refer to fixed connection, detachable connection or integrated connection; mechanical connection or electrical connection; directly connected or indirectly connected through an intermediary, or connection between two elements. Those ordinarily skilled in the art shall understand the specific meanings of the terms above in this invention as the case may be.

In addition, the technical features involved in different embodiments of this invention described below can be mutually combined as long as they do not conflict.

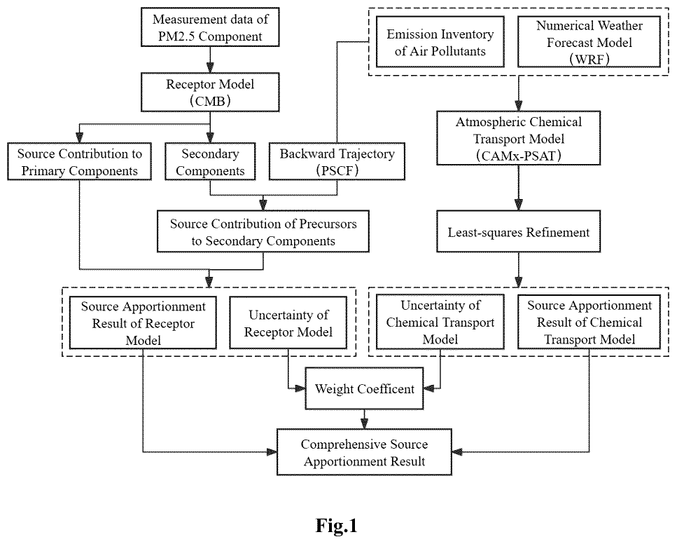

See FIG. 1, which shows the flow chart of a method for comprehensively apportioning the source contribution to PM_{2.5} based on the receptor model and the chemical transport model provided in an exemplary embodiment of this invention, including the receptor model calculation step, the chemical transport model calculation step and the comprehensive source apportionment step;

The said receptor model calculation step comprises the following sub-steps:

According to the receptor points, setting up a simulation grid, compiling a grid-based emission inventory, and simulating the meteorological field using the mesoscale weather forecast model WRF;

Implementing receptor sampling analysis to obtain the component concentration of PM_{2.5} at the receptor points, and inputting the component concentration into the receptor model CMB to obtain the primary pollution contribution of different sources to PM_{2.5};

Based on the said meteorological field, using the potential source contribution calculation method PSCF in the backward trajectory model to obtain the spatial range having potential source influence on the concentration of PM_{2.5}; calculating the emission proportions of sulfur dioxide, nitrogen oxides and volatile organic compounds from different sources in the spatial range;

Distributing the pollution source contribution to the secondary component (SO₄²⁻, NO₃⁻, SOC, etc.) concentration of PM_{2.5} according to the said emission proportion to obtain the secondary pollution contributions of different sources to PM_{2.5};

Adding the primary pollution contribution of different sources to PM_{2.5} and the secondary pollution contribution of different sources to PM_{2.5}, so as to obtain the overall pollution contribution of different sources to PM_{2.5}, i.e., the source apportionment result SRⱼ of the receptor model;

Calculating the first uncertainty σⱼ^{SR} of the source apportionment result SRⱼ of the receptor model;

The said chemical transport model calculation step comprises the following sub-steps: According to the receptor points, setting up a simulation grid, compiling a grid-based emission inventory, and simulating the meteorological field using the mesoscale numerical weather forecast model WRF;

Inputting the said meteorological field and the grid-based emission inventory into the chemical transport model CAMx/PSAT to obtain the initial simulated concentration of the PM_{2.5} components and the pollution source contribution value of PM_{2.5};

Performing optimization solution based on the least-squares error between the simulated result and the observed result of the chemical transport model of the PM_{2.5} component concentration at the receptor point, so as to obtain the correction factor of the simulated result of the chemical transport model;

Using the said correction factor to correct the value of the pollution source contribution to PM_{2.5} simulated by the chemical transport model, so as to obtain the corrected value of the pollution source contribution to PM_{2.5}, i.e., the apportionment result of the chemical transport model SAⱼ;

Calculating the second uncertainty σⱼ^{SA} of the apportionment result SAⱼ of the chemical transport model;

The said comprehensive source apportionment step comprises the following sub-steps: The uncertainty range of the model apportionment result represents the error range of the result, so the range of the first uncertainty σⱼ^{SR} and the range of the second uncertainty σⱼ^{SA} are normalized according to the principle that the uncertainty range is inversely proportional to the weight coefficient, so as to obtain their respective weight coefficient. The calculation formula is as follows:

$$\{ \begin{matrix}{w}_{j}^{SR} = \frac{span ( {σ}_{j}^{SA} )}{span ( {σ}_{j}^{SR} ) + span ( {σ}_{j}^{SA} )} \\ {w}_{j}^{SA} = \frac{span ( {σ}_{j}^{SR} )}{span ( {σ}_{j}^{SR} ) + span ( {σ}_{j}^{SA} )}\end{matrix}$$

Wherein, wⱼ^{SR} and wⱼ^{SA} respectively represent the weight coefficient of the source apportionment result of the receptor model and the weight coefficient of the source apportionment result of the chemical transport model, and “span” represents the uncertainty range calculated;

Calculating the comprehensive source apportionment result Sⱼ based on the source apportionment result SRⱼ of the receptor model, weight coefficient wⱼ^{SR} of the source apportionment result of the receptor model, apportionment result SAⱼ of the chemical transport model and weight coefficient wⱼ^{SA} of the source apportionment result of the chemical transport model:

$${S}_{j} = {w}_{j}^{SR} \times {SR}_{j} + {w}_{j}^{SA} \times {SA}_{j} .$$

Specifically, in this exemplary embodiment, first setting up a simulation grid, compiling a grid-based emission inventory, and simulating the meteorological field using WRF; using the receptor model CMB to apportion the pollution contribution of different sources to the primary components of PM2.5; using the potential source contribution PSCF to identify the spatial range of the pollution influences, and calculating the pollution contribution to the secondary components of PM_{2.5} based on the emission proportions of sulfur dioxide, nitrogen oxides and volatile organic compounds from different sources in the range above; simulating the component concentration of PM_{2.5} and the pollution contribution of the emission sources by using the chemical transport model CAMx/PSAT; monitoring and simulating the least-squares error of the component concentrations based on the receptor points, calculating the correction factor of the simulated result of CAMx/PSAT model, and correcting the source apportionment results of CAMx/PSAT; normalizing the uncertainties of the receptor model CMB and the chemical transport model CAMx/PSAT to obtain the weight coefficients of the two source apportionment results, so as to obtain the comprehensive source apportionment results.

In this exemplary embodiment:
(1) Calculating the weight coefficients of the two source apportionment results based on the normalization of the uncertainties of the receptor model and the chemical transport model, so as to obtain the comprehensive source apportionment result; the effects are as follows:
(1-1) Compared with the prior art, it is very complicated to calculate the multi-model data similarity to obtain the model weight coefficient. In this exemplary embodiment, the model weight coefficient is obtained through the uncertainty, in which the uncertainty is used to evaluate the error of the source apportionment result of the model (which can be directly extracted from the database for use in some cases) as a parameter that can be easily calculated after the source apportionment result of each model is calculated, so it is unnecessary to calculate additional parameters in the common process (additional parameters are required to be calculated in calculating the data similarity in the prior art) to reduce the amount of data calculated;
(1-2) At the same time, the uncertainty of the model apportionment result represents the resulting error, and the uncertainty can be a positive deviation or negative deviation in some cases, so the uncertainty range (i.e., the difference between the positive deviation and the negative deviation of the uncertainty) can characterize the possible deviation range of the results. After the first uncertainty range and the second uncertainty range are normalized based on the principle that the uncertainty range is inversely proportional to the weight coefficient (a large range corresponds to a small weight, while a small range corresponds to a large weight), so as to obtain their respective weight coefficient, thus improving the accuracy and reliability of the source apportionment result of PM2.5;
(1-3) In addition, the weight coefficients of all sources (industrial sources, traffic sources, domestic sources, etc.) are determined based on their respective uncertainty results to provide higher purposefulness and refinement (because different sources have different pollutant components and characteristics, the receptor model and the chemical transport model vary in terms of applicability), and more refined traceability results can be obtained through the differentiated source contribution weight coefficients.
(2) For the receptor model, the potential source contribution analysis method is used to specify the influence range of the pollution transport of PM_{2.5}, and the source contribution to the secondary component of PM_{2.5} is distributed and calculated based on the precursor emissions proportions of all resources based on the range above, thus optimizing the source apportionment result of the secondary component of the receptor model; for the chemical transport model, the least-squares error between the simulated result and the monitoring data of the PM_{2.5} components is used to optimize the model and thus correct the source apportionment result of the chemical transport model.

The specific embodiments of the receptor model calculation step, chemical transport model calculation step and comprehensive source apportionment step are detailed below. In terms of the receptor model calculation step:

Preferably, in an exemplary embodiment, setting a simulation grid according to the said receptor points, comprising:

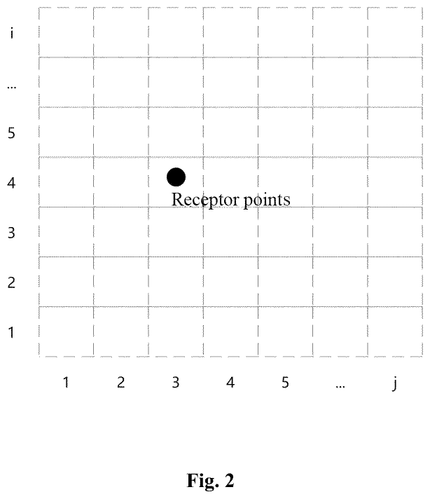

The receptor points are the points to be traced for sources, the simulation grid is a WRF simulation grid, and the simulation grid of the study area should cover the receptor points, as shown in FIG. 2;

Compiling a grid-based emission inventory as said above, comprising:

Inputting the products listed on the local anthropogenic emissions or other public emission inventory calculated by using the factor accounting method based on the data, including environmental statistics, pollutant discharge permit and enterprise research into the emission inventory processing model SMOKE, so as to obtain the grid-based pollutant discharge inventory suitable for the chemical transport model CAMx/PSAT.

Wherein, SMOKE (Sparse Matrix Operator Kernel Emissions) is a pollution source emission inventory processing tool developed by the University of North Carolina, USA. It adopts the high-performance computing sparse matrix algorithm to distribute the annual average emissions of pollutants in the emission source inventory based on time, space and species, so as to make a grid-based hourly emission inventory file that meets the requirements of air quality model. The air quality model (CAMx) is an Eulerian regional photochemical sparse model, which allows considering the tropospheric air pollutants (ozone, particulate matter, atmospheric toxic substances) from the urban scale to the continental spatial scale as “a whole atmosphere”, and the PSAT source apportionment technology tracks the source contribution to the particulate matter contribution predicted through the source regions and/or categories.

More preferably, in an exemplary embodiment, simulating the meteorological field using the mesoscale numerical weather forecast model WRF as said above, comprising:

Inputting the re-analysis meteorological data, local terrain elevation and data of underlying surface covered by land into WRF, simulating the meteorological field for a period of time, and verifying the simulated result and optimizing its parameters based on the observed data of the meteorological station.

Wherein, the WRF (The Weather Research and Forecasting Model) includes topographic data processing, ground and sounding data processing, numerical simulation, post-processing modules, etc., and applies to various weather conditions in the region ranging from tens of meters to thousands of kilometers.

Preferably, in an exemplary embodiment, implementing receptor sampling analysis to obtain the component concentration of PM_{2.5} at the receptor points as said above, comprising:

Collecting the particulate filter membrane using an atmospheric sampler, the chemical element analysis, carbon analysis and ion analysis of the samples are completed using the inductively coupled plasma mass spectrometer (ICP-MS), inductively coupled plasma spectrometry (ICP-OES), ion chromatography and thermal/optical carbon analyzer;

The analyzed components comprise one or more of the chemical elements below: Li, Be, Na, P, K, Sc, As, Rb, Y, Mo, Cd, Sn, Sb, Cs, La, V, Cr, Mn, Co, Ni, Cu, Zn, Ce, Sm, W, TI, Pb, Bi, Th, U, Zr, Al, Sr, Mg, Ti, Ca, Fe, Ba, Si; one or more of the carbon components below: TC, OC and EC, and one or more of the ion components below: Na⁺, Mg²⁺, Ca²⁺, K⁺, NH₄+, SO₄²⁻, Cl⁻ and NO₃⁻;

The said different sources comprise power sources, industrial sources, traffic sources, domestic sources, agricultural sources and other sources.

Preferably, in an exemplary embodiment, based on the said meteorological field, using the potential source contribution calculation method PSCF in the backward trajectory model to obtain the spatial range having potential source influence on the concentration of PM_{2.5}; calculating the emission proportions of sulfur dioxide, nitrogen oxides and volatile organic compounds from different sources in the spatial range, as said above, comprise:

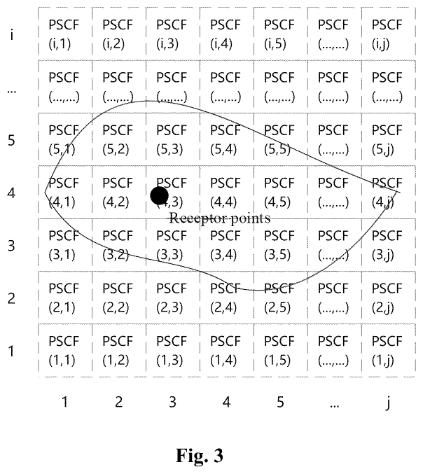

Converting the simulated result WRFOUT file of WRF into a format that can be recognized by HYSPLIT model through HYSPLIT model pre-processing tool, then inputting the converted meteorological data into HYSPLIT model for simulation to obtain a backward trajectory for a period of time, and finally obtaining the spatial range having potential source influence and the PSCF value of each grid through PSCF method based on the backward trajectory, as shown in FIG. 3;

HYSPLIT model is a professional model jointly developed by the National Oceanic and Atmospheric Administration (NOAA) and the Air Resources Laboratory (ARL) to calculate and analyze the transport and diffusion trajectory of air pollutants. The HYSPLIT is one of the most widely used atmospheric transport and diffusion models in the atmospheric science community. The common application is backward trajectory analysis, which is used to determine the source of air mass and establish the source-receptor relationship. The PSCF method uses the backward trajectory to calculate the conditional probability function that is used to describe the geographical location and spatial distribution of possible source area.

PSCF is a method to characterize the pollution contribution of each grid to the receptor points by using the probability of airflow traceability. This method can divide the study area into several small horizontal grids based on longitudes and latitudes and set the concentration threshold of the pollutants to compare whether the pollutant concentrations at the traceability points in the grids are higher than the threshold (for example, the concentration of PM2.5 is higher than 35 μg/m³) and thus determine the number of pollution trajectory points in the grids. The PSCF value is the ratio of the number (mᵢⱼ) of pollution trajectory points passing through the grid (i, j) in the study area to the number (nᵢⱼ) of all trajectory points passing through the grid, namely PSCF=mᵢⱼ/nᵢⱼ.

The total amount of emission of a certain type of pollutant from a certain source is calculated by

$$E = \frac{\sum ( {PSCF}_{ij} \times {E}_{ij} )}{\sum {PSCF}_{ij}}$$

Wherein, Eᵢⱼ represents the emission of the pollutants from the source in (i, j) grid. Accordingly, the emissions of sulfur dioxide, nitrogen oxides and volatile organic compounds from different sources that affect the receptor points and the proportion of the total emissions of such pollutants from all sources in the spatial range can be calculated.

Preferably, in an exemplary embodiment, calculating the first uncertainty σⱼ^{SR} of the source apportionment result SRⱼ of the receptor mode, as said above, comprising:

The first uncertainty σⱼ^{SR} is calculated based on the observed error and the emission inventory error of the PM_{2.5} components:

$${σ}_{j}^{SR} = {\sum }_{i = 1}^{N} ( {p}_{i} {σ}_{i , j}^{obs} + {p}_{i}^{′} {σ}_{i , j}^{emi} )$$

Wherein, pᵢ is the concentration proportion of the primary component i of PM_{2.5}; σ_{i,j}^{obs} is the observed error of the PM_{2.5} component, which is specifically reflected on the observed uncertainty of the PM_{2.5} component i and source j; p′ᵢ is the concentration proportion of the secondary component i of PM_{2.5}; σ_{i,j}^{emi} is the emission inventory error, which is specifically reflected in the uncertainty of the precursor emission inventory of the secondary component i and source j of PM_{2.5};

Among them, the observed error σ_{i,j}^{obs} of the PM_{2.5} component is calculated based on the ratio of the maximum permissible error to the typical observed concentration of the component observation instrument; the emission inventory error σ_{i,j}^{emi} is calculated based on the uncertainty of the multiscale air pollution emission inventory.

More specifically, the uncertainty of the PM_{2.5} components is observed based on the ratio of the maximum allowable error to the typical observed concentration of the component observation instrument, and the uncertainties of the OC/EC component analyzed with the carbon component analyzer, SO4²⁻, NO₃⁻ and NH₄⁺, etc. analyzed with the water-solute ion analyzer and the continental crust elements analyzed with the element analyzers are respectively calculated. If the same observation instrument of the PM_{2.5} components is used, the maximum allowable error of the instrument can be directly used, otherwise the uncertainties can be re-calculated according to the actual data sources.

The emission inventory error σ_{i,j}^{emi} is also the uncertainty of the precursor emission inventory of the secondary component of PM_{2.5}, which is closely related to the adopted source emission inventory. China's air pollutant emission inventory is generally the multiscale air pollutant emission inventory MEIC developed by Tsinghua University in China, and the uncertainty range of the estimated emissions in the inventory within a 95% confidence interval is certain. If the MEIC emission inventory is used, the uncertainty can be determined by reference to the study results of Li et al (2017) and Zhao et al (2013) on the uncertainty of China's anthropogenic emission inventory, for example, the uncertainty range of the MEIC emissions estimated within 95% confidence interval is shown in the table below, i.e., the uncertainty of NH³ is ±153%, and the uncertainty of NMVOC is ±78% (Kurokawa et al, 2013). If the compiled localized emission inventory is used, the actual method and data used should be compiled according to the inventory to facilitate the calculation of the uncertainty of the localized emission inventory.

TABLE 1
Examples of Uncertainty of Emission Inventory (Unit: %)

| | Power | Industrial | Traffic | Domestic | |
|---|---|---|---|---|---|
| | sources | sources | sources | sources | Average |
| SO₂ | −27-59 | −22-27 | −21-41 | −46-51 | −15-26 |
| NOₓ | −19-15 | −32-90 | −30-56 | −37-101 | −15-35 |
| PM_{2.5} | −34-62 | −15-85 | −30-50 | −54-99 | −15-63 |
| NMVOC | ±78 | ±78 | ±78 | ±78 | ±78 |
| NH³ | ±153 | ±153 | ±153 | ±153 | ±153 |
| BC | −68-574 | −49-117 | −71-77 | −53-240 | −28-126 |
| OC | −76-273 | −41-141 | −68-86 | −57-136 | −42-114 |

In terms of the chemical transport model calculation step, “According to the receptor points, setting up a simulation grid, compiling a grid-based emission inventory, and simulating the meteorological field using the mesoscale numerical weather forecast model WRF” is the same as the corresponding sub-steps of the receptor model calculation step and will not be repeated here, but in terms of other steps:

Preferably, in an exemplary embodiment, inputting the said meteorological field and the grid-based emission inventory into the chemical transport model CAMx/PSAT, and the main parameterization schemes are specifically as follows: The initial field and boundary condition are ICBCPREP, the meteorological chemical mechanism is CB05, the aqueous chemical mechanism is RADM, the aerosol scheme is CF scheme, the secondary organic chemical scheme is SOAP, the aerosol thermodynamic equilibrium model is ISORROPIA, the dry deposition parameterization scheme is ZHANG03, the horizontal advection scheme is PPM scheme, and the vertical diffusion scheme is standard K theory.

Preferably, in an exemplary embodiment, performing optimization solution based on the least-squares error between the simulated result and the observed result of the chemical transport model of the PM_{2.5} component concentration at the receptor point, so as to obtain the correction factor of the simulated result of the chemical transport model, as said above, comprising:

Taking the least-squares error between the CAMx simulated concentration and observed concentration of the PM_{2.5} component at the receptor point as the target function to obtain the correction factor corresponding to the minimum error, with the target function below:

$$min {\sum }_{i = 1}^{N} [ \frac{{( {C}_{i}^{obs} - {C}_{i}^{sim} - {\sum }_{j = 1}^{M} {SA}_{i , j}^{b a s e} ( {R}_{j} - 1 ) )}^{2}}{{σ}_{i , obs}^{2} + {σ}_{i , sim}^{2}} ]$$

$$s . t . 0.1 \leq {R}_{j} \leq 20$$

Wherein, Rⱼ is the correction factor of the model apportionment result of the source j; Cᵢ^{obs}, Cᵢ^{sim} are respectively the observed concentration value and the simulated concentration value of the component i; SA_{i,j}^{base} is the simulated contribution concentration of the initial model of the component i and the source j; σ_{i,obs}, σ_{i,sim} are respectively the uncertainties of the observed concentration and the simulated concentration of the component i;

Inputting the observed result of the PM_{2.5} component and the initial source contribution of PSAT, etc. into the target function, and performing a non-linear optimization solution on the target function to obtain the correction factor R of the model apportionment result; using the step-by-step iterative optimization calculation method in the solution procedure, that is, the initial iterative optimization step size is 0.5, and the optimization step size of 0.01 is adopted after the preliminary determination of range.

Specifically, in terms of the correction factor of the simulated result of the chemical transport model, the formula adopted in the prior art involves the parameter, i.e., normalized model error, the calculation process of which is complicated, thus leading to a more complicated optimization iteration process of the whole correction factor. However, in this application, the optimization formula is more concise, and the iterative calculation method for optimization is more efficient. Though calculation, accurate data can also be obtained.

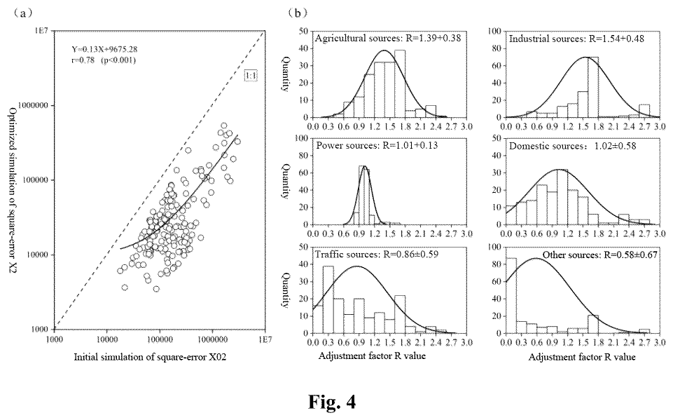

As shown in FIG. 4, through the application of the exemplary embodiments, the least-squares error of the optimized CAMx/PSAT decreased by about 85% compared with the initial model error. The distribution range of the PM_{2.5} pollution source correction factor R values is between 0.06 and 2.60, and the average values for the power sources, industrial sources, traffic sources, domestic sources, agricultural sources and other sources are respectively 1.01±0.13, 1.54±0.48, 0.86±0.598, 1.02±0.58, 1.39±0.3 and 0.58±0.67. A larger difference between R and 1 suggests a higher uncertainty, that is, the uncertainties for the agricultural sources, industrial sources, traffic sources and other sources are relatively higher. Different pollution sources have different cumulative distribution functions of R values, which are related to the pollutant emission characteristics.

Preferably, in an exemplary embodiment, as said above, using the said correction factor to correct the value of the pollution source contribution to PM_{2.5} simulated by the chemical transport model, so as to obtain the corrected value of the pollution source contribution to PM_{2.5}, i.e., the apportionment result of the chemical transport model SAⱼ, with the calculation formula comprising:

$${SA}_{i , j}^{adj} = {R}_{j} \times {SA}_{i , j}^{b a s e}$$

$${SA}_{j} = {\sum }_{i = 1}^{N} {SA}_{i , j}^{a d j}$$

Wherein, SA_{i,j}^{adj} is the simulated contribution concentration of the correction model of the PM_{2.5} component i and source j; SAⱼ is the simulated source apportionment result of the PM_{2.5} model of the source j.

TABLE 2
Examples of CAMx/PSAT optimization result of source apportionment of PM_{2.5}

| | | CAMx/PSAT initial result | | CAMx/PSAT optimization result | |
|---|---|---|---|---|---|
| Pollution | Correction | Concentration | Contribution | Concentration | Contribution |
| sources | factor | (μg/m³) | (%) | (μg/m³) | (%) |
| Power | 1.01 ± 0.13 | 4.43 | 5.46 | 4.46 | 5.22 |
| sources | | | | | |
| Industrial | 1.54 ± 0.48 | 16.22 | 19.98 | 24.11 | 28.23 |
| sources | | | | | |
| Traffic | 0.86 ± 0.598 | 7.03 | 8.65 | 6.48 | 7.59 |
| sources | | | | | |
| Domestic | 1.02 ± 0.58 | 44.65 | 54.97 | 39.49 | 46.23 |
| sources | | | | | |
| Agricultural | 1.39 ± 0.3 | 7.32 | 9.01 | 10.03 | 11.74 |
| sources | | | | | |
| Other | 0.58 ± 0.67 | 1.57 | 1.93 | 0.84 | 0.98 |
| sources | | | | | |
| Total | — | 81.22 | 100.00 | 85.41 | 100.00 |
| contribution | | | | | |
| value | | | | | |

Preferably, in an exemplary embodiment, calculating the second uncertainty σⱼ^{SA} of the apportionment result SAⱼ of the chemical transport model, as said above, with the calculation formula comprising:

$${σ}_{j}^{SA} = {\sum }_{i = 1}^{N} {p}_{i} f ( {σ}_{i , j}^{emi} )$$

Wherein, pᵢ is the concentration proportion of the PM_{2.5} component i; σ_{i,j}^{emi} is the uncertainty of the emission inventory of the PM_{2.5} component i and source j, and f(σ_{i,j}^{emi}) is the uncertainty of the simulated result derived from that of the emission inventory.

Specifically, in the exemplary embodiment, f(σ_{i,j}^{emi}) can be calculated by using the prior art, for example, f(σ_{i,j}^{emi}) uncertainty analysis can be implemented by building a simplified model (such as the HDDM-SRSM method) by reference to the study of Kai Yang et al (2020) to replace the original atmospheric chemical transport model, so as to improve the efficiency of uncertainty analysis; the random sampling method of Monte Carlo is used to transfer the uncertainty of the simplified model, and the uncertainty of the chemical transport model can be quantified based on 95% confidence interval of the simulated results of the PM_{2.5} components. In the method used by Kai Yang et al (2020), the first-order sensitivity coefficient of each variable is calculated and obtained by using the sensitivity analysis tool HDDM of the atmospheric chemical transport model CMAQ, and the atmospheric chemical transport model CAMx used in this exemplary embodiment also has a HDDM sensitivity analysis tool, so the HDDM-SRSM method can be used to significantly reduce the amount of simulated calculation of the atmospheric chemical transport model, thus simplifying the calculation of the second uncertainty σⱼ^{SA}.

In terms of the comprehensive source apportionment step, its specific embodiment is as follows:

The uncertainty range of the model apportionment result represents the error range of the result, so the range of the first uncertainty σⱼ^{SR} and the range of the second uncertainty σⱼ^{SA} are normalized according to the principle that the uncertainty range is inversely proportional to the weight coefficient, so as to obtain their respective weight coefficient. The calculation formula is as follows:

$$\{ \begin{matrix}{w}_{j}^{SR} = \frac{span ( {σ}_{j}^{SA} )}{span ( {σ}_{j}^{SR} ) + span ( {σ}_{j}^{SA} )} \\ {w}_{j}^{SA} = \frac{span ( {σ}_{j}^{SR} )}{span ( {σ}_{j}^{SR} ) + span ( {σ}_{j}^{SA} )}\end{matrix}$$

Wherein, wⱼ^{SR} and wⱼ^{SA} respectively represent the weight coefficient of the source apportionment result of the receptor model and the weight coefficient of the source apportionment result of the chemical transport model, and “span” represents the uncertainty range calculated; if the uncertainty is [−50%, 150%], the uncertainty range is 200%;

Calculating the comprehensive source apportionment result Sⱼ based on the source apportionment result SRⱼ of the receptor model, weight coefficient wⱼ^{SR} of the source apportionment result of the receptor model, apportionment result SAⱼ of the chemical transport model and weight coefficient wⱼ^{SA} of the source apportionment result of the chemical transport model:

$${S}_{j} = {w}_{j}^{SR} \times {SR}_{j} + {w}_{j}^{SA} \times {SA}_{j} .$$

However, in this exemplary embodiment, the comprehensive source apportionment results are shown in Table 3.

TABLE 3
Examples of comprehensive source apportionment weights and results of PM_{2.5}

| | | | | | Comprehensive source | |
|---|---|---|---|---|---|---|
| | CAMx/PSAT | | | | apportionment results | |
| Pollution | optimization results | | CMB results | | Contribution | Contribution |
| sources | Uncertainty | Weight | Uncertainty | Weight | (μg/m³) | (%) |
| Power | −18%~65% | 0.63 | −66%~73% | 0.37 | 5.10 | 6.06 |
| sources | | | | | | |
| Industrial | −20%~126% | 0.52 | −64%~92% | 0.48 | 22.91 | 27.25 |
| sources | | | | | | |
| Traffic | −20%~90% | 0.58 | −68~82% | 0.42 | 11.83 | 14.08 |
| sources | | | | | | |
| Domestic | −32%~155% | 0.53 | −99%~114% | 0.47 | 36.61 | 43.53 |
| sources | | | | | | |
| Agricultural | −115%~275% | 0.43 | −145%~145% | 0.57 | 6.47 | 7.69 |
| sources | | | | | | |
| Other | −11%~67% | 0.56 | −41%~58% | 0.44 | 0.87 | 1.04 |
| sources | | | | | | |

Based on the same inventive concept as this exemplary embodiment above, another exemplary embodiment of this invention provides an electronic device, including a memory unit and a processing unit, wherein the said memory unit stores the computer instructions that are executable on the said processing unit while the said processing unit executes the steps in a method for comprehensively apportioning the source contribution to PM_{2.5} based on the receptor model and the chemical transport model when executing the said computer instructions.

The electronic device is represented in the form of a general computing device. The components of the electronic device may include, but are not limited to, at least one processing unit above, at least one memory unit above, and a bus connecting different system components (including the memory unit and the processing unit).

Wherein, the said memory unit stores program codes, and the said program codes can be executed by the said processing unit, so that the said processing unit can execute the steps of various exemplary embodiments of this invention given in the “Exemplary Methods” above of this Specifications. For example, the said processing unit can execute the method given in FIG. 1.

The memory unit may include readable media in the form of the volatile memory unit, such as a random access memory unit (RAM) 3201 and/or a cache memory unit, and may further include a read-only memory unit (ROM).

The memory unit may also include a program/utility with a set of (at least one) program modules, and such program modules include, but are not limited to, an operating system, one or more application programs or programs, other program modules and program data, and each or some combination of these examples may include the implementation of the network environment.

The bus can represent one or more of several bus structures, including a memory unit or a memory unit controller, a peripheral bus, an accelerated graphics port, a processing unit, or a local bus using any bus structure in a variety of bus structures.

The electronic device may also communicate with one or more external devices (e.g., keyboard, pointing device, Bluetooth device, etc.), one or more devices that enable users to interact with the electronic device, and/or any device that enables the electronic device to communicate with one or more other computing devices (e.g., router, modem, etc.). Such communication can be carried out through the input/output (I/O) interface. Moreover, the electronic device may also communicate with one or more networks (such as local area network (LAN), wide area network (WAN) and/or public network such as the Internet) through the network adapter. The network adapter communicates with other modules through the bus and the electronic device. It should be understood that other hardware and/or software modules can be used in combination with the electronic device, including but not limited to: microcode, device drives, redundant processing units, external disk drive arrays, RAID systems, tape drives and data backup storage system.

Through the description above, those skilled in this art could easily understand that the exemplary embodiments described here can be implemented through software or the combination of software and hardware. Therefore, the technical proposal given according to this exemplary embodiment can be implemented in the form of a software product. The said software product can be stored in non-volatile memory storage (such as a CD-ROM, a U disk, or a mobile hard disk) or on the network, including several instructions that enable a computing device (which may be a personal computer, a server, a terminal device or a network device, etc.) to execute the method given according to this exemplary embodiment).

Based on the same inventive concept as this exemplary embodiment above, another exemplary embodiment of this invention provides a memory medium to store the computer instructions, and the steps in the method for comprehensively apportioning the source contribution to PM2.5 based on the receptor model and the chemical transport model are executed when the said computer instructions are executed.

Based on such understanding, the technical protocol in the embodiment can be implemented in the form of software products (program products) essentially or in any part contributing to the prior art or any part of the technical protocol. The computer software product is stored in a memory medium, including all or some of the steps of several instructions that enable a computer device (which may be a personal computer, a server, or a network device) to execute the method given in each embodiment of this invention.

The said program products may be any combination of one or more readable media. The readable medium may refer to a readable signal medium or a readable memory medium. The readable memory medium includes (but is not limited to) an electrical, magnetic, optical, electromagnetic, infrared or semiconductor system, equipment or device, or any combination hereof. More specific examples of the readable memory medium (a non-exhaustive list) include an electrical connection with one or more wires, a portable disk, a hard disk, a random access memory (RAM), a read-only memory (ROM), an erasable programmable read-only memory (EPROM or flash memory), an optical fiber, a portable compact read-only memory (CD-ROM), an optical memory device, a magnetic memory device, or any combination hereof.

The computer-readable signal medium may include a data signal that is propagated in a baseband or as part of the carrier wave, in which the readable program codes are carried. The data signal propagated in this way may be (including but not limited to) an electromagnetic signal, an optical signal or any appropriate combination hereof. The readable signal medium may also refer to any readable medium other than the readable memory medium, and the readable medium can send, propagate or transmit a program used by or with the instruction-executing system, equipment or device.

The program codes contained by the readable medium may be transmitted through any appropriate medium, including but not limited to wireless, wired, optical cable, RF, or any appropriate combination hereof.

Any combination of one or more programming languages can be used to write codes for executing the operations of this invention, and the said programming languages include object-oriented programming languages, such as Java and C++, and also include conventional procedural programming languages, such as “C” languages or similar programming languages. The program codes may be completely executed on the user computing device, partially executed on the user computing device, executed as an independent software package, partially executed respectively on the user computing device and the remote computing device, or completely executed on the remote computing device or server. When a remote computing device is involved, the remote computing device may be connected to the user computing device through any kind of network, such as a local area network (LAN) or wide area network (WAN), or may be connected to an external computing device (for example, through the Internet by the Internet service provider).

Obviously, the embodiments above are only examples given to make a clear explanation but not to limit the modes of implementation. Ordinary technical personnel in the relevant field can make other changes or variations based on the above description. Here, it is unnecessary and impossible to exhaust all the modes of implementation. However, the obvious changes or variations derived therefrom still fall within the scope of protection created by this invention.

What is claimed:

1. A method for comprehensively apportioning the source contribution to fine particular mater (PM_{2.5}) based on the receptor model and the chemical transport model, characterized by comprising receptor model calculation steps, the chemical transport model calculation steps and the comprehensive source apportionment steps;
The said receptor model calculation steps described herein comprise the following sub-steps:
According to the receptor points, set up a simulation grid, compile a grid-based emission inventory, and simulate the meteorological field using the mesoscale numerical weather forecast model WRF;
Implementing receptor sampling analysis to obtain the component concentration of PM_{2.5} at the receptor pointsr, and inputting the component concentration into the receptor model CMB to obtain the primary pollution contribution of different sources to PM_{2.5};
Based on the said meteorological field, using the potential source contribution calculation method PSCF in the backward trajectory model to obtain the spatial range having potential source influence on the concentration of PM_{2.5}, and calculating the emission proportions of sulfur dioxide, nitrogen oxides and volatile organic compounds from different sources in spatial range;
Distributing the pollution source contribution to the secondary component concentration of PM_{2.5} according to the said emission proportion to obtain the secondary pollution contributions of different sources to PM_{2.5};
Adding the primary pollution contribution of different sources to PM_{2.5} and the secondary pollution contributions of different sources to PM_{2.5}, so as to obtain the overall pollution contribution of different sources to PM_{2.5}, i.e., the source apportionment result SRⱼ of the receptor model;
Calculating the first uncertainty σⱼ^{SA} of the source apportionment result SRⱼ of the receptor model;
The said chemical transport model calculation step comprises the following sub-steps:
According to the receptor points, setting up a simulation grid, compiling a grid-based emission inventory, and simulating the meteorological field using the mesoscale numerical weather forecast model WRF;
Inputting the said meteorological field and the grid-based emission inventory into the chemical transport model CAMx/PSAT to obtain the initial simulated concentration of PM_{2.5} components and the pollution source contribution value of PM_{2.5};
Performing optimization solution based on the least-squares error between the simulated result and the observed result of the chemical transport model of PM_{2.5} component concentration at the receptor point, so as to obtain the correction factor of the simulated result of the chemical transport model;
Using the said correction factor to correct the value of the pollution source contribution to PM_{2.5} simulated by the chemical transport model, so as to obtain the corrected value of the pollution source contribution to PM_{2.5}, i.e., the apportionment result of the chemical transport model SAⱼ;
Calculating the second uncertainty σⱼ^{SA} of the apportionment result SAⱼ of the chemical transport model;
The said comprehensive source apportionment step comprises the following sub-steps:
As the uncertainty range of the model apportionment result represents the error range of the result, the ranges of the first uncertainty σⱼ^{SR} and the range of the second uncertainty σⱼ^{SA} are normalized according to the principle that the uncertainty range is inversely proportional to the weight coefficient, so as to obtain their respective weight coefficient. The calculation formula is as follows:
$$\{ \begin{matrix}{w}_{j}^{SR} = \frac{span ( {σ}_{j}^{SA} )}{span ( {σ}_{j}^{SR} ) + span ( {σ}_{j}^{SA} )} \\ {w}_{j}^{SA} = \frac{span ( {σ}_{j}^{SR} )}{span ( {σ}_{j}^{SR} ) + span ( {σ}_{j}^{SA} )}\end{matrix}$$
Wherein, wⱼ^{SR} and wⱼ^{SA} represent respectively the weight coefficient of the source apportionment result of the receptor model and the weight coefficient of the source apportionment result of the chemical transport model, and “span” represents the uncertainty range calculated;
Calculating the comprehensive source apportionment result Sⱼ based on the source apportionment result SRⱼ of the receptor model, weight coefficient wⱼ^{SR} of the source apportionment result of the receptor model, apportionment result SAⱼ of the chemical transport model and weight coefficient wⱼ^{SR} of the source apportionment result of the chemical transport model;
$${S}_{j} = {w}_{j}^{SR} \times {SR}_{j} + {w}_{j}^{SA} \times {SA}_{j} .$$

2. According to the method for comprehensively apportioning the source contribution to fine particular matter based on the receptor model and the chemical transport model that is mentioned in claim 1, which is characterized by setting up a simulation grid according to the receptor points as said above, comprising:
The receptor points refer to the locations where traceability is required, the simulation grid is a WRF simulation grid, and the simulation grid of the study area should cover the receptor points;
Compiling a grid-based emission inventory as said above, comprising:
The locally-derived anthropogenic emissions or other publicly available emissions inventory products, calculated by using the factor accounting method based on the data including environmental statistics, pollutant discharge permit and enterprise surveys, are input into the emission inventory processing model SMOKE, so as to obtain the grid-based pollutant emissions inventories suitable for the chemical transport model CAMx/PSAT.

3. According to the method for comprehensively apportioning the source contribution to PM_{2.5} based on the receptor and chemical transport model mentioned in claim 1, which is characterized by using the mesoscale numerical weather forecast model WRF to simulate the meteorological field, comprising:
Inputting the re-analysis meteorological data, local terrain elevation and data of the underlying surface covered by land into WRF, simulating the meteorological field for a period of time, and verifying the simulated results and optimizing its parameters based on the observed data from the meteorological station.

4. According to the method for comprehensively apportioning the source contribution to PM_{2.5} based on the receptor and chemical transport model mentioned in claim 1, which is characterized by conducting receptor sampling analysis to obtain the component concentration of PM_{2.5} at the receptor points, comprising:
Using an atmospheric sampler to collect particulate filter membrane samples, the chemical element analysis, carbon analysis and ion analysis of the samples are completed using the inductively coupled plasma mass spectrometer (ICP-MS), inductively coupled plasma spectrometry (ICP-OES), ion chromatography and thermal/optical carbon analyzer;
The analyzed components comprise one or more of the chemical elements below: Li, Be, Na, P, K, Sc, As, Rb, Y, Mo, Cd, Sn, Sb, Cs, La, V, Cr, Mn, Co, Ni, Cu, Zn, Ce, Sm, W, TI, Pb, Bi, Th, U, Zr, Al, Sr, Mg, Ti, Ca, Fe, Ba, Si; one or more of the carbon components below: TC, OC and EC, and one or more of the ion components below: Na⁺, Mg²⁺, Ca²⁺, K⁺, NH₄⁺, SO₄²⁻, Cl⁻ and NO₃⁻;
The said different sources comprise the power sources, industrial sources, traffic sources, domestic sources, agricultural sources and other sources.

5. According to the method for comprehensively apportioning the source contribution to PM_{2.5} based on the receptor model and the chemical transport model that is mentioned in claim 1, which is characterized by based on the said meteorological field, using the potential source contribution calculation method PSCF in the backward trajectory model to obtain the spatial range having potential source influence on the concentration of PM_{2.5}; calculating the emission proportions of sulfur dioxide, nitrogen oxides and volatile organic compounds from different sources in the spatial range, as said above, comprise:
Converting the simulated result WRFOUT file of WRF into a format that can be recognized by the HYSPLIT model through the HYSPLIT model pre-processing tool, then inputting the converted meteorological data into the HYSPLIT model for simulation to obtain a backward trajectory for a period of time, and finally obtaining the spatial range having potential source influence and the PSCF value of each grid through PSCF method based on the backward trajectory;
PSCF is a method to characterize the pollution contribution of each grid to the receptor points by using the probability of airflow traceability. This method can divide the study area into several small horizontal grids based on longitudes and latitudes and set the concentration threshold of the pollutants to compare whether the pollutant concentrations at the traceability points in the grids are higher than the threshold (for example, the concentration of PM_{2.5} is higher than 35 μg/m³) and thus determine the number of pollution trajectory points in the grids. The PSCF value is the ratio of the number (mᵢⱼ) of pollution trajectory points passing through the grid (i, j) in the study area to the number (nᵢⱼ) of all trajectory points passing through the grid, namely PSCF=mᵢⱼ/nᵢⱼ. The total amount of emission of a certain type of pollutant from a certain source is calculated by
$$E = \frac{\sum ( {PSCF}_{ij} \times {E}_{ij} )}{\sum {PSCF}_{ij}}$$
Wherein, Eᵢⱼ represents the emission of the pollutants from the source in (i, j) grid. Accordingly, the emissions of sulfur dioxide, nitrogen oxides and volatile organic compounds from different sources that affect the receptor points and the proportion of the total emissions of such pollutants from all sources in the spatial range can be calculated.

6. According to the method for comprehensively apportioning the source contribution to PM_{2.5} based on the receptor model and the chemical transport model mentioned in claim 1, which is characterized by calculating the first uncertainty σⱼ^{SR} of the source apportionment result SRⱼ of the receptor model, as said above, comprising:
The first uncertainty σⱼ^{SR} is calculated based on the observed error and the emission inventory error of the PM2.5 components:
$${σ}_{j}^{SR} = {\sum }_{i = 1}^{N} ( {p}_{i} {σ}_{i , j}^{obs} + {p}_{i}^{′} {σ}_{i , j}^{emi} )$$
Wherein, pᵢ is the concentration proportion of the primary component i of PM_{2.5}; σ_{i,j}^{obs} is the observed error of the PM_{2.5} component, which is specifically reflected on the observed uncertainty of the PM_{2.5} component i and source j; p′ᵢ is the concentration proportion of the secondary component i of PM_{2.5}; σ_{i,j}^{emi} is the emission inventory error, which is specifically reflected in the uncertainty of the precursor emission inventory of the secondary component i and source j of PM_{2.5};
Among them, the observed error σ_{i,j}^{obs} of the PM_{2.5} component is calculated based on the ratio of the maximum permissible error to the typical observed concentration of the component observation instrument; the emission inventory error σ_{i,j}^{emi} can be calculated based on the uncertainty of the multiscale air pollution emission inventory.

7. According to the method for comprehensively apportioning the source contribution to PM_{2.5} based on the receptor model and the chemical transport model that is mentioned in claim 1, which is characterized by inputting the said meteorological field and the grid-based emission inventory into the chemical transport model CAMx/PSAT, and the main parameterization schemes are specifically as follows: The initial field and boundary condition are ICBCPREP, the meteorological chemical mechanism is CB05, the aqueous chemical mechanism is RADM, the aerosol scheme is CF scheme, the secondary organic chemical scheme is SOAP, the aerosol thermodynamic equilibrium model is ISORROPIA, the dry deposition parameterization scheme is ZHANG03, the horizontal advection scheme is PPM scheme, and the vertical diffusion scheme is standard K theory.

8. According to the method for comprehensively apportioning the source contribution to PM_{2.5} based on the receptor model and the chemical transport model that is mentioned in claim 1, which is characterized by performing optimization solution based on the least-squares error between the simulated result and the observed result of the chemical transport model of the PM_{2.5} component concentration at the receptor point, so as obtain the correction factor of the simulated result of the chemical transport model, as said above, comprising:
Taking the least-squares error between the CAMx simulated concentration and observed concentration of the PM_{2.5} component at the receptor point as the target function to obtain the correction factor corresponding to the minimum error, with the target function below:
$$min {\sum }_{i = 1}^{N} [ \frac{{( {C}_{i}^{obs} - {C}_{i}^{sim} - {\sum }_{j = 1}^{M} {SA}_{i , j}^{b a s e} ( {R}_{j} - 1 ) )}^{2}}{{σ}_{i , obs}^{2} + {σ}_{i , sim}^{2}} ]$$
$$s . t . 0.1 \leq {R}_{j} \leq 20$$
Wherein, Rⱼ is the correction factor of the model apportionment result of the source j; Cᵢ^{obs}, Cᵢ^{sim} are respectively the observed concentration value and the simulated concentration value of the component i; SA_{i,j}^{base} is the simulated contribution concentration of the initial model of the component i and the source j; σ_{i,obs}, σ_{i,sim} are respectively the uncertainties of the observed concentration and the simulated concentration of the component i;
Inputting the observed result of the PM_{2.5} component and the initial source contribution of PSAT, etc. into the target function, and performing a non-linear optimization solution on the target function to obtain the correction factor R of the model apportionment result; using the step-by-step iterative optimization calculation method in the solution procedure, that is, the initial iterative optimization step size is 0.5, and the optimization step size of 0.01 is adopted after the preliminary determination of range.

9. According to the method for comprehensively apportioning the source contribution to PM_{2.5} based on the receptor model and the chemical transport model that is mentioned in claim 1, which is characterized by using the said correction factor to correct the value of the pollution source contribution to PM_{2.5} simulated by the chemical transport model, so as to obtain the corrected value of the pollution source contribution to PM_{2.5}, i.e., the apportionment result of the chemical transport model SAⱼ, with the calculation formula comprising:
$${SA}_{i , j}^{adj} = {R}_{j} \times {SA}_{i , j}^{b a s e}$$
$${SA}_{j} = {\sum }_{i = 1}^{N} {SA}_{i , j}^{a d j}$$
Wherein, SA_{i,j}^{adj} is the simulated contribution concentration of the correction model of the PM_{2.5} component i and source j; SAⱼ is the simulated source apportionment result of the PM_{2.5} model of the source j.

10. According to the method for comprehensively apportioning the source contribution to PM_{2.5} based on the receptor model and the chemical transport model that is mentioned in claim 1, which is characterized by calculating the second uncertainty σⱼ^{SA} of the apportionment result SAⱼ of the chemical transport model, as said above, with the calculation formula comprising:
$${σ}_{j}^{SA} = {\sum }_{i = 1}^{N} {p}_{i} f ( {σ}_{i , j}^{emi} )$$
Wherein, pᵢ is the concentration proportion of the PM_{2.5} component i; σ_{i,j}^{emi} is the uncertainty of the emission inventory of the PM_{2.5} component i and source j, and f(σ_{i,j}^{emi}) is the uncertainty of the simulated result derived from that of the emission inventory.
